(19) United States
(12) Patent Application Publication
(10) Pub. No.: US 2020/0318080 A1
(43) Pub. Date: Oct. 8, 2020

(54) METHODS AND COMPOSITIONS FOR TREATING EQUINE CONDITIONS USING RECOMBINANT SELF-COMPLEMENTARY ADENO-ASSOCIATED VIRUS

(71) Applicants: COLORADO STATE UNIVERSITY RESEARCH FOUNDATION, FORT COLLINS, CO (US); CALIMMUNE, INC, TUCSON, AZ (US); THE UNIVERSITY OF NORTH CAROLINA AT CHAPEL HILL, CHAPEL HILL, NC (US)

(72) Inventors: Laurie R. Goodrich, Bellvue, CO (US); C. Wayne McILwraith, Loveland, CO (US); Jeffrey S. Bartlett, Columbus, OH (US); Richard Jude Samulski, Chapel Hill, NC (US)

(21) Appl. No.: 16/326,601

(22) PCT Filed: Aug. 18, 2017
(86) PCT No.: PCT/US17/47572

§ 371 Date: Feb. 19, 2019

Related U.S. Application Data
(60) Provisional application No. 62/377,281, filed on Aug. 19, 2016.

(51) Int. Cl. C12N 7/00; C07K 14/545; C12N 15/86; A61K 48/00; A61K 35/16; A61K 38/20; A61P 19/02; A61K 31/573; A61K 31/728

(52) U.S. Cl. CPC C12N 7/00; C07K 14/545; C12N 15/86; A61K 48/0075; A61K 35/16; A61K 38/2006; C12N 2750/14142; A61P 19/02; A61K 31/573; A61K 31/728; C12N 2750/14143; C12N 2750/14131; A61K 48/005

(57) ABSTRACT

Methods and compositions for treating symptoms of conditions such as but not limited to osteoarthritis in horses. The methods may feature direct intraarticular injection of a recombinant self-complementary adeno-associated virus (sc-rAAV) with a vector adapted to express a modified IL-1 Ra peptide. The methods of the present invention may express a therapeutically effective amount of the modified IL-1 Ra peptide so as to ameliorating symptoms associated with the condition being treated.

### CROSS REFERENCE

This application claims priority to U.S. Provisional Patent Application No. 62/377,281 filed Aug. 19, 2016, the specification(s) of which is/are incorporated herein in their entirety by reference.

### GOVERNMENT SUPPORT

This invention was made with government support under Grant No. AR054903 awarded by National Institutes of Health. The government has certain rights in the invention.

### REFERENCE TO SEQUENCE LISTING

Applicant asserts that the information recorded in the form of an Annex C/ST.25 text file submitted under Rule 13ter.1(a), entitled CLAIM_16_01_PCT_Sequence_Listing_ST25.txt, is identical to that forming part of the international application as filed. The content of the sequence listing is incorporated herein by reference in its entirety.

### FIELD OF THE INVENTION

The present invention relates to gene therapy and compositions for gene therapy, more particularly to recombinant self-complementary adeno-associated virus (sc-rAAV) and methods of treating conditions or symptoms of conditions using sc-rAAV.

### BACKGROUND OF THE INVENTION

IL-1 is a powerful mediator of both chondrocytic chondrolysis and suppression of matrix synthesis by chondrocytes. Together, these two processes are highly destructive to cartilage. IL-1 has also been shown to inhibit chondrogenesis but at the same time promote certain aspects of the osteogenic differentiation that could help account for the formation of osteophytes and sclerosis of sub-chondral bone. In studying cartilage recovered from human joints with OA, the production of IL-1 by chondrocytes was found to be highly elevated and sustained in an autocrine fashion. Moreover, the cells did not produce IL-1Ra. This suggests enhanced autocrine and paracrine activation of chondrocytes by IL-1 in the absence of its major physiological inhibitor during OA. Enhanced responsiveness of chondrocytes to IL-1 in OA was also indicated by increased expression of the type I IL-1 receptor, the signaling receptor, on OA chondrocytes. The local production and consumption of IL-1 by chondrocytes may help explain why concentrations of IL-1 in synovial fluid tend to be low, even in OA. Also, genetic analyses have identified single nucleotide polymorphisms (SNPs) in the human gene encoding IL-1Ra (IL1RN) and regulatory elements that correlate with the incidence and severity of certain types of OA.

Targeted drug delivery is a major problem for the intra-articular treatment of joint diseases. Molecules of all sizes, as well as particles, are rapidly removed from joints via the lymphatics, subsynovial capillaries, or both. This makes it difficult to achieve sustained, therapeutic doses of anti-OA drugs in joints. To address this, small molecules can be delivered systemically, but proteins are difficult to deliver in this fashion because of size-dependent constraints in crossing the fenestrated endothelium of the synovial capillaries. Moreover, systemic delivery exposes non-target sites to high doses of the therapeutic, leading to unwanted side-effects. The rapid egress of proteins from joints, with half-lives typically of a few hours, makes intra-articular delivery potentially ineffective. As an example, recombinant IL-1Ra (Kineret, Amgen Biologicals) is delivered by daily subcutaneous injection in effort to treat symptoms of RA. However, daily delivery fails to maintain therapeutic serum levels of IL-1Ra between injections (Evans et al., 1996, Human Gene Therapy, 7:1261-1290; Evans et al., 2005, PNAS 102 (24): 8698-8703). Some studies have used ex vivo gene transfer for introducing IL-1Ra to treat OA. However, these approaches are laborious and have not seemed to provide long-term gene expression (Frisbie et al., 2002, Gene Therapy 9(1): 12-20). Also, several studies describe the use of a dual variable domain-immunoglobulin (DVD-Ig) targeting IL-1alpha and IL-1beta (e.g., ABT-981) for treating osteoarthritis (Kamath et al., 2011, Osteoarthritis and Cartilage 19S1:S64; Wang et al., 2015, Osteoarthritis and Cartilage 23:A398-399; Wang et al., 2014, Osteoarthritis and cartilage 22:S462-S463; Lacy et al., 2015, mAbs 7(3):605-619; Wu et al., 2009, mAbs 1(4):339-347; Wang et al., 2014, Scientific Abstracts SAT0448 pg. 756; Goss et al., 2014, Scientific Abstracts SAT0447 pg. 755-756; US 2015/0050238; Wang et al., 2014 ACR/ARHP Annual Meeting Abstract Number 2237; Wang et al., 2015 ACR/ARHP Annual Meeting Abstract Number 318). However, these peptides require repeated systemic introduction (e.g., 4 doses every 2 weeks or 3 doses every 4 weeks, e.g., by subcutaneous injection or intravenous infusion) because of the relatively short half-life (Wang et al., 2015, Osteoarthritis and Cartilage 23:A398-399; Wang et al., 2014, Osteoarthritis and cartilage 22:S462-S463; Evans et al., 2005, PNAS 102 (24): 8698-8703).

The present invention features methods and compositions for delivering a therapeutic gene product (e.g., IL-1Ra) in a sustained manner to a location of interest, e.g., joints, in horses. The present invention also features methods and compositions for treating symptoms of conditions such as but not limited to osteoarthritis. The present invention also features methods and compositions for providing a horse a therapeutically effective amount of a therapeutic gene product (e.g., IL-1Ra). The methods and compositions may feature a recombinant self-complementary adeno-associated virus (sc-rAAV), wherein the sc-rAAV comprises an engineered capsid and a vector (e.g., a sc-rAAV vector) packaged within the capsid. The vector may comprise a transgene (e.g., a nucleotide sequence encoding a protein of interest, e.g., a therapeutic gene product, e.g., IL-1Ra or a modified version thereof) operably linked to a promoter (e.g., a constitutive promoter). The therapeutic gene product may be delivered to a location of interest, e.g., a joint. For example, for treating osteoarthritis, the sc-rAAV may be introduced into cells (e.g., chondrocytes, synoviocytes, etc.) in a joint via direct intraarticular injection. The present invention is not limited to the aforementioned conditions, nor the location of interest (e.g., joint).

It is noted that Goodrich et al. (Molecular Therapy-Nucleic Acids, 2013, 2:e70) generally discloses a method of treating osteoarthritis using scAAV-delivered IL-1Ra. However, Goodrich et al. does not specifically identify or enable any particular IL-1Ra sequence, e.g., an IL-1Ra sequence according to the present invention. In particular, the field of gene therapy is an unpredictable area wherein one cannot assume that any particular gene sequence for a protein of interest will be efficiently expressed.

### SUMMARY OF THE INVENTION

The present invention features a recombinant self-complementary adeno-associated virus (sc-rAAV). In some embodiments, the sc-rAAV comprises an engineered AAV capsid and a vector packaged within the capsid, wherein the vector comprises a modified IL-1Ra gene operably linked to a promoter and the modified IL-1Ra gene is at least 95% identical to SEQ ID NO: 2. In some embodiments, the promoter comprises a CMV promoter. In some embodiments, the engineered capsid comprises at least a portion of serotype AAV2 and at least a portion of serotype AAV6. In some embodiments, the engineered capsid comprises at least a portion of serotype AAV1, AAV2, AAV3, AAV4, AAV5, AAV6, AAV7, AAV8, AAV9, AAV10, AAV11, or a combination thereof. In some embodiments, the vector further comprises SV40 and bovine growth hormone (bGH) polyadenylation sequences. In some embodiments, the vector further comprises SV40 splice donor (SD) and splice acceptor (SA) sites. In some embodiments, the vector comprises sc-rAAV2.5Hu-IL-1Ra. In some embodiments, the sc-rAAV is part of a composition.

In some embodiments, the sc-rAAV comprises an engineered AAV capsid and a vector packaged within the capsid, wherein the vector comprises a modified IL-1Ra gene operably linked to a promoter and the modified IL-1Ra gene encodes IL-1Ra protein according to SEQ ID NO: 6 or SEQ ID NO: 7.

The present invention features a method of providing a horse in need thereof (e.g., a horse diagnosed with or at risk for osteoarthritis) a therapeutically effective amount of interleukin-1 receptor agonist (IL-1Ra) peptide. In some embodiments, the method comprises introducing into a location of interest (e.g., via intraarticular injection) a composition comprising a recombinant self-complementary adeno-associated virus (sc-rAAV) according to the present invention. The sc-rAAV transduces the vector into cells in the location of interest, wherein the modified IL-1Ra gene is expressed so as to provide the horse with the therapeutically effective amount of said IL-1Ra peptide.

The present invention also features a method of ameliorating symptoms of osteoarthritis in a horse. In some embodiments, the method comprises introducing into a location of interest (e.g., via direct intraarticular injection) a composition comprising a recombinant self-complementary adeno-associated virus (sc-rAAV) according to the present invention. The sc-rAAV transduces the vector into cells in the location of interest, wherein the modified IL-1Ra gene is expressed so as to provide the horse with an amount of IL-1Ra peptide effective for ameliorating symptoms associated with osteoarthritis.

The present invention also features a method of repairing cartilage in a horse in need thereof (e.g., a horse diagnosed with or at risk for developing osteoarthritis). In some embodiments, the method comprises introducing into a location of cartilage (e.g., via direct intraarticular injection) a composition comprising a recombinant self-complementary adeno-associated virus (sc-rAAV) according to the present invention. The sc-rAAV transduces the vector into cells in the location of cartilage, wherein the modified IL-1Ra gene is expressed so as to provide the horse with IL-1Ra peptide effective for repairing cartilage.

The present invention also features a method of providing interleukin-1 receptor agonist (IL-1Ra) peptide to an area of inflammation. In some embodiments, the method comprises introducing into a location of inflammation (e.g., via intraarticular injection) a composition comprising a recombinant self-complementary adeno-associated virus (sc-rAAV) according to the present invention. The sc-rAAV transduces the vector into cells in the location of inflammation, wherein the modified IL-1Ra gene is expressed so as to provide the cells in the location of inflammation a therapeutically effective amount of IL-1Ra peptide effective for reducing inflammation.

In some embodiments, the location of interest is a joint, synovium, subsynovium, joint capsule, tendon, ligament, cartilage, or peri-articular muscle of the horse. In some embodiments, the cells are chondrocytes, synoviocytes, or a combination thereof.

In some embodiments, the method is performed a second time at a time point after a time when the method is performed first. In some embodiments, the time point is at least 3 months. In some embodiments, the method further comprises co-introducing a secondary therapy (e.g., a glucocorticoid, hyaluronan, platelet-rich plasma, recombinant, horse IL-1Ra, or a combination thereof) to the location of interest in combination with the composition.

The present invention also features a method of delivering IL-1Ra peptide to a chondrocyte or synoviocyte. In some embodiments, the method comprises contacting the chondrocyte or synoviocyte with a recombinant self-complementary adeno-associated virus (sc-rAAV) according to the present invention, e.g., an engineered adeno-associated virus (AAV) capsid comprising at least a portion of serotype 2 and at least a portion of serotype 6 and a vector packaged within the capsid, wherein the vector comprises a modified IL-1Ra gene operably linked to a CMV promoter and the modified IL-1Ra gene is at least 95% identical to SEQ ID NO: 2. The sc-rAAV transduces the vector into the chondrocyte or synoviocyte and the modified IL-1Ra gene is expressed to as to provide IL-1Ra peptide to the chondrocyte or synoviocyte.

For the aforementioned methods and compositions (e.g., a method of providing a horse in need thereof a therapeutically effective amount of interleukin-1 receptor agonist (IL-1Ra) peptide, a method of ameliorating symptoms of osteoarthritis or rheumatoid arthritis in a horse, a method of delivering IL-1Ra peptide to a chondrocyte or synoviocyte, a composition comprising a recombinant self-complementary adeno-associated virus (sc-rAAV), a recombinant self-complementary adeno-associated virus (sc-rAAV) vector comprising a modified IL-1Ra gene operably linked to a CMV promoter, a method of repairing cartilage in a canine in need thereof, a method of providing interleukin-1 receptor agonist (IL-1Ra) peptide to an area of inflammation, etc.), the modified IL-1Ra gene may be at least 95% identical SEQ ID NO: 2 and encode IL-1Ra according to SEQ ID NO: 6 or SEQ ID NO: 7.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1 shows Plasmid p-trsKS-cmv-opt-eq-il1-ra, which contains a modified cDNA encoding IL-1Ra protein under control of the CMV promoter. A modified sequence for the equine IL-1ra gene was removed from a non-expression vector using Notl and Agel restriction enzyme sites (NEB, Ipswich, Mass.), and ligated into a pTRs-ks mammalian expression vector obtained from the UNC Vector Core (Chapel Hill, N.C.) containing a CMV promoter. Ligations were performed using T4 ligase as per manufacturer's instructions (NEB). Constructs were transformed into DH10 Electrocompetent cells (Invitrogen) and evaluated for ITR sites using Smal enzyme sites (NEB). Once these sites were confirmed, the constructs were then transformed into SURE cells (Invitrogen), and evaluated again for ITR sites. Constructs are ampicillin resistant. The total size of the construct is 5.754 kb in length.

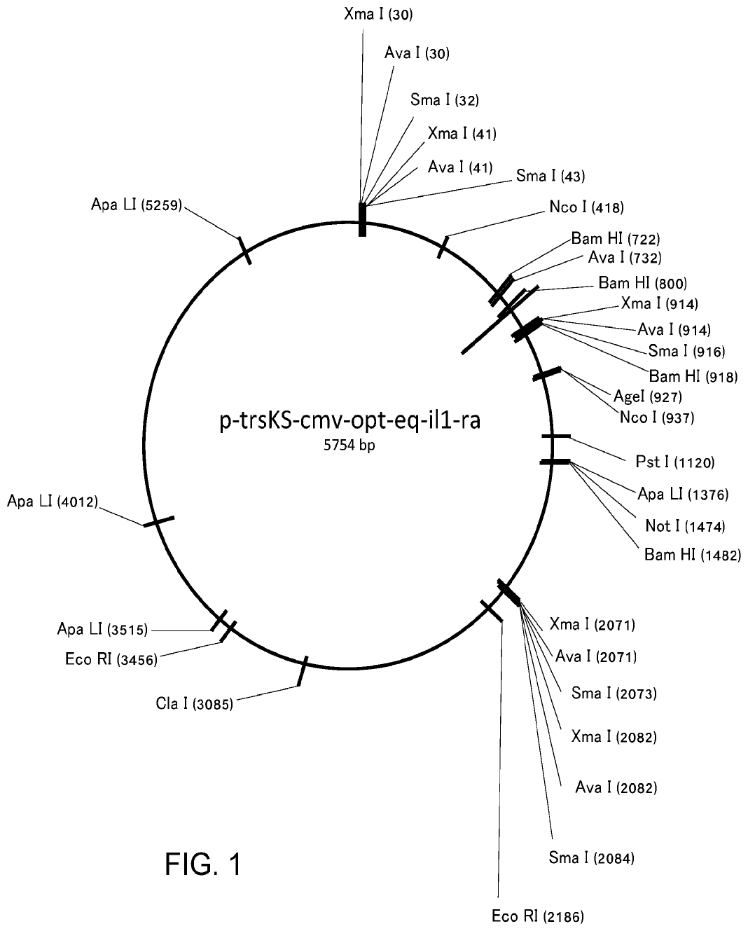

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety herein by reference.

All publications, patent applications, patents, and other references mentioned herein are incorporated by reference in their entirety.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Adeno-Associated Virus (AAV), Recombinant AAV (rAAV), and Recombinant Self-Complementary AAV (Sc-rAAV):

AAV is a small virus (20 nm) in the family Parvoviridae. AAV is not known to cause disease. AAV has recently been used to gene therapy for a variety of reasons including that it has been shown to have low immunogenicity, the ability to effectively transduce non-dividing cells, and the ability to infect a variety of cell and tissue types. Recombinant AAV (rAAV) does not contain native viral coding sequences. Recombinant AAV DNA is packaged into the viral capsid as a single stranded molecule about 4600 nucleotides in length. Following infection of the cell by the virus, the molecular machinery of the cell converts the single DNA strand into a double-stranded form. Only the double stranded DNA form is useful to the proteins of the cell that transcribe the contained gene or genes into RNA. Self-complementary AAV (sc-rAAV) is an engineered form of rAAV that can form an intra-molecular double stranded DNA template. Thus, upon infection, the two complementary halves of sc-rAAV will associate to form one double stranded DNA unit that is ready for immediate replication and synthesis.

Expression:

The translation of a nucleic acid sequence into a protein. Proteins may be expressed and remain intracellular, become a component of the cell surface membrane, or be secreted into the extracellular matrix or medium.

Operably Linked:

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

Pharmaceutically Acceptable Vehicles:

Pharmaceutically acceptable carriers (vehicles), e.g., solutions, may be conventional but are not limited to conventional vehicles. For example, E. W. Martin, Remington's Pharmaceutical Sciences, Mack Publishing Co., Easton, Pa., 15th Edition (1975) and D. B. Troy, ed. Remington: The Science and Practice of Pharmacy, Lippincott Williams & Wilkins, Baltimore Md. and Philadelphia, Pa., 21^{st} Edition (2006) describe compositions and formulations suitable for pharmaceutical delivery of one or more therapeutic compounds or molecules. In general, the nature of the carrier will depend on the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like, for example sodium acetate or sorbitan monolaurate.

Preventing, Treating, Managing, or Ameliorating a Condition:

“Preventing” a disease may refer to inhibiting the full development of a condition. “Treating” may refer to a therapeutic intervention that ameliorates a sign or symptom of a disease or pathological condition after it has begun to develop. “Managing” may refer to a therapeutic intervention that does not allow the signs or symptoms of a disease or condition to worsen. “Ameliorating” may refer to the reduction in the number or severity of signs or symptoms of a disease or condition.

Sequence Identity:

The identity (or similarity) between two or more nucleic acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN may be used to compare nucleic acid sequences, while BLASTP may be used to compare amino acid sequences. If the two compared sequences share homology, then the designated output file will present those regions of homology as aligned sequences. If the two compared sequences do not share homology, then the designated output file will not present aligned sequences. The BLAST-like alignment tool (BLAT) may also be used to compare nucleic acid sequences (Kent, Genome Res. 12:656-664, 2002). BLAT is available from several sources, including Kent Informatics (Santa Cruz, Calif.) and on the Internet (genome.ucsc.edu). Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth.

Therapeutically Effective Amount:

A quantity of a specified agent sufficient to achieve a desired effect in a subject being treated with that agent. Such agents may include IL-1Ra. For example, a therapeutically effective amount of IL-1Ra may be an amount sufficient to prevent, treat, or ameliorate symptoms of osteoarthritis. The therapeutically effective amount of an agent useful for preventing, ameliorating, and/or treating a subject will be dependent on the subject being treated, the type and severity of the affliction, and the manner of administration of the therapeutic composition.

Transduced:

A transduced cell is a cell into which a nucleic acid molecule has been introduced by molecular biology techniques. As used herein, the term transduction encompasses all techniques by which a nucleic acid molecule might be introduced into such a cell, including transfection with viruses or viral vectors, transformation with plasmid vectors, and introduction of naked DNA by electroporation, lipofection, and particle gun acceleration. Such cells are sometimes called transformed cells.

Vector:

A nucleic acid molecule as introduced into a host cell, thereby producing a transformed host cell. A vector may include nucleic acid sequences that permit it to replicate in a host cell, such as an origin of replication. A vector may lack the nucleic acid sequences that permit it to replicate in a host cell. A vector may also include a gene of interest, one or more selectable marker genes, other genetic elements known in the art, or any other appropriate insert.

### DETAILED DESCRIPTION OF THE INVENTION

The present invention features methods and compositions for delivering a therapeutic gene product (e.g., IL-1Ra) in a sustained manner to a location of interest, e.g., a joint. The present invention also features methods and compositions for treating symptoms of conditions such as but not limited to osteoarthritis. The present invention also features methods and compositions for providing an individual (e.g., a horse) a therapeutically effective amount of a therapeutic gene product (e.g., IL-1Ra). The methods and compositions may feature a recombinant self-complementary adeno-associated virus (sc-rAAV), wherein the sc-rAAV comprises an engineered capsid and a vector (an sc-rAAV vector) packaged within the capsid. The vector may comprise a transgene (e.g., a nucleotide sequence encoding a protein of interest, e.g., a therapeutic gene product, e.g., IL-1Ra or a modified version thereof) operably linked to a promoter (e.g., a constitutive promoter).

As previously discussed, the present invention features compositions comprising a recombinant self-complementary adeno-associated virus (sc-rAAVs) vector. A non-limiting example of a sc-rAAV vector is shown in SEQ ID NO: 1 of Table 1 below. The sc-rAAV vector of SEQ ID NO: 1 comprises a modified IL-1Ra gene (the sequence within SEQ ID NO: 1 that encodes IL-1Ra is underlined). The sc-rAAV vector is not limited to SEQ ID NO: 1. In some embodiments, the sc-rAAV vector comprises a nucleic acid sequence for IL-1Ra according to SEQ ID NO: 2. The present invention is not limited to SEQ ID NO: 2.

The sc-rAAV vectors comprise a nucleic acid that encodes a peptide of interest. In some embodiments, the nucleic acid is at least 90% identical to SEQ ID NO: 2. In some embodiments, the nucleic acid is at least 92% identical to SEQ ID NO: 2. In some embodiments, the nucleic acid is at least 94% identical to SEQ ID NO: 2. In some embodiments, the nucleic acid is at least 95% identical to SEQ ID NO: 2. In some embodiments, the nucleic acid is at least 96% identical to SEQ ID NO: 2. In some embodiments, the nucleic acid is at least 97% identical to SEQ ID NO: 2. In some embodiments, the nucleic acid is at least 98% identical to SEQ ID NO: 2. In some embodiments, the nucleic acid is at least 99% identical to SEQ ID NO: 2. Non-limiting examples of such nucleic acid sequences can be found in Table 1 below. For example, SEQ ID NO: 3 is a sequence for a modified IL-1Ra that is about 98% identical to SEQ ID NO: 2; SEQ ID NO: 4 is a sequence for a modified IL-1Ra that is about 99% identical to SEQ ID NO: 2; and SEQ ID NO: 5 is a sequence for a modified IL-1Ra that is about 95% identical to SEQ ID NO: 2 (note that the bold letters in Table 1 are nucleotide substitutions as compared to SEQ ID NO: 2, and the codon underlined).

TABLE 1

| SEQ | | |
|---|---|---|
| ID | | |
| NO: | DESCRIPTION | SEQUENCE |
| 1 | Equine Modified IL-1ra p- | ctgcgcgctcgctcgctcactgaggccgcccgggcaaagcccgggcgt |
| | trs-KS Expression Vector | cgggcgacctttggtcgcccggcctcagtgagcgagcgagcgcgcaga |
| | Sequence with CMV | gagggagtggggttcggtacccgttacataacttacggtaaatggccc |
| | promotor & ampicillin | gcctggctgaccgcccaacgacccccgcccattgacgtcaataatgac |
| | resistance | gtatgttcccatagtaacgccaatagggactttccattgacgtcaatg |
| | | ggtggagtatttacggtaaactgcccacttggcagtacatcaagtgta |
| | | tcatatgccaagtacgccccctattgacgtcaatgacggtaaatggcc |
| | | cgcctggcattatgcccagtacatgaccttatgggactttcctacttg |
| | | gcagtacatctacgtattagtcatcgctattaccatggtgatgcggtt |
| | | ttggcagtacatcaatgggcgtggatagcggtttgactcacggggatt |
| | | tccaagtctccaccccattgacgtcaatgggagtttgttttggcacca |
| | | aaatcaacgggactttccaaaatgtcgtaacaactccgccccattgac |
| | | gcaaatgggcggtaggcgtgtacggtgggaggtctatataagcagagc |
| | | tcgtttagtgaaccgtcagatcgcctggagacgccatccacgctgttt |
| | | tgacctccatagaagacaccgggaccgatccagcctccggactctaga |
| | | ggatccggtactcgaggaactgaaaaaccagaaagttaactggtaagt |
| | | ttagtctttttgtcttttatttcaggtcccggatccggtggtggtgca |
| | | aatcaaagaactgctcctcagtggatgttgcctttacttctaggcctg |
| | | tacggaagtgttacttctgctctaaaagctgcggaattgtacccgcgg |
| | | cccgggatccggtaccggtgccaccatggagatcagacgcagaagcgt |
| | | gcgccacctgatcagcctgctgctgttcctgctgtacagcgagaccgc |
| | | ctgccaccccctgggcaagaggccctgcaagatgcaggccttcagaat |
| | | ctgggacgtgaatcagaaaaccttctacatgcgcaacaatcagctggt |
| | | cgctggctacctgcaggagagcaacaccaagctgcaagagaagatcga |
| | | cgtggtgcccatcgagcccgacgccctgttcctgggcctgcacggcag |
| | | aaagctgtgcctggcctgcgtgaagtccggcgacgagatcagattcca |
| | | gctggaggccgtgaacatcaccgacctgagcaagaacaaggaggagaa |
| | | caagcgcttcaccttcatcagaagcaacagcggccctaccacctcctt |
| | | cgagagcgccgcctgccccggctggttcctgtgcaccgcccaggaggc |
| | | cgacagacctgtctccctgaccaacaagcccaaagaatccttcatggt |
| | | gaccaagttctacctgcaagaggatcagtgagcggccgcgggatccag |
| | | acatgataagatacattgatgagtttggacaaaccacaactagaatgc |
| | | agtgaaaaaaatgctttatttgtgaaatttgtgatgctattgctttat |
| | | ttgtaaccattataagctgcaataaacaagttaacaacaacaattgca |
| | | ttcattttatgtttcaggttcagggggaggtgtgggaggttttttagt |
| | | cgactagagctcgctgatcagcctcgactgtgccttctagttgccagc |
| | | catctgttgtttgcccctcccccgtgccttccttgaccctggaaggtg |
| | | ccactcccactgtcctttcctaataaaatgaggaaattgcatcgcatt |
| | | gtctgagtaggtgtcattctattctggggggtggggtggggcaggaca |
| | | gcaagggggaggattgggaagacaatagcaggctggggagagatccta |
| | | gtttccatgctctaggagcatggctacgtagataagtagcatggcggg |
| | | ttaatcattaactacaaggaacccctagtgatggagttggccactccc |
| | | tctctgcgcgctcgctcgctcactgaggccgggcgaccaaaggtcgcc |
| | | cgacgcccgggctttgcccgggcggcctcagtgagcgagcgagcgcgc |
| | | cagctggcgtaatagcgaagaggcccgcaccgatcgcccttcccaaca |
| | | gttgcgcagcctgaatggcgaatggaattccagacgattgagcgtcaa |
| | | aatgtaggtatttccatgagcgtttttcctgttgcaatggctggcggt |
| | | aatattgttctggatattaccagcaaggccgatagtttgagttcttct |
| | | actcaggcaagtgatgttattactaatcaaagaagtattgcgacaacg |
| | | gttaatttgcgtgatggacagactcttttactcggtggcctcactgat |
| | | tataaaaacacttctcaggattctggcgtaccgttcctgtctaaaatc |
| | | cctttaatcggcctcctgtttagctcccgctctgattctaacgaggaa |
| | | agcacgttatacgtgctcgtcaaagcaaccatagtacgcgccctgtag |
| | | cggcgcattaagcgcggcgggtgtggtggttacgcgcagcgtgaccgc |
| | | tacacttgccagcgccctagcgcccgctcctttcgctttcttcccttc |
| | | ctttctcgccacgttcgccggctttccccgtcaagctctaaatcgggg |
| | | gctccctttagggttccgatttagtgctttacggcacctcgaccccaa |
| | | aaaacttgattagggtgatggttcacgtagtgggccatcgccctgata |
| | | gacggtttttcgccctttgacgttggagtccacgttctttaatagtgg |
| | | actcttgttccaaactggaacaacactcaaccctatctcggtctattc |
| | | ttttgatttataagggattttgccgatttcggcctattggttaaaaaa |
| | | tgagctgatttaacaaaaatttaacgcgaattttaacaaaatattaac |
| | | gtttacaatttaaatatttgcttatacaatcttcctgtttttggggct |
| | | tttctgattatcaaccggggtacatatgattgacatgctagttttacg |
| | | attaccgttcatcgattctcttgtttgctccagactctcaggcaatga |
| | | cctgatagcctttgtagagacctctcaaaaatagctaccctctccggc |
| | | atgaatttatcagctagaacggttgaatatcatattgatggtgatttg |
| | | actgtctccggcctttctcacccgtttgaatctttacctacacattac |
| | | tcaggcattgcatttaaaatatatgagggttctaaaaatttttatcct |
| | | tgcgttgaaataaaggcttctcccgcaaaagtattacagggtcataat |
| | | gtttttggtacaaccgatttagctttatgctctgaggctttattgctt |
| | | aattttgctaattctttgccttgcctgtatgatttattggatgttgga |
| | | attcctgatgcggtattttctccttacgcatctgtgcggtatttcaca |
| | | ccgcatatggtgcactctcagtacaatctgctctgatgccgcatagtt |
| | | aagccagccccgacacccgccaacacccgctgacgcgccctgacgggc |
| | | ttgtctgctcccggcatccgcttacagacaagctgtgaccgtctccgg |
| | | gagctgcatgtgtcagaggttttcaccgtcatcaccgaaacgcgcgag |
| | | acgaaagggcctcgtgatacgcctatttttataggttaatgtcatgat |
| | | aataatggtttcttagacgtcaggtggcacttttcggggaaatgtgcg |
| | | cggaacccctatttgtttatttttctaaatacattcaaatatgtatcc |
| | | gctcatgagacaataaccctgataaatgcttcaataatattgaaaaag |
| | | gaagagtatgagtattcaacatttccgtgtcgcccttattcccttttt |
| | | tgcggcattttgccttcctgtttttgctcacccagaaacgctggtgaa |
| | | agtaaaagatgctgaagatcagttgggtgcacgagtgggttacatcga |
| | | actggatctcaacagcggtaagatccttgagagttttcgccccgaaga |
| | | acgttttccaatgatgagcacttttaaagttctgctatgtggcgcggt |
| | | attatcccgtattgacgccgggcaagagcaactcggtcgccgcataca |
| | | ctattctcagaatgacttggttgagtactcaccagtcacagaaaagca |
| | | tcttacggatggcatgacagtaagagaattatgcagtgctgccataac |
| | | catgagtgataacactgcggccaacttacttctgacaacgatcggagg |
| | | accgaaggagctaaccgcttttttgcacaacatgggggatcatgtaac |
| | | tcgccttgatcgttgggaaccggagctgaatgaagccataccaaacga |
| | | cgagcgtgacaccacgatgcctgtagcaatggcaacaacgttgcgcaa |
| | | actattaactggcgaactacttactctagcttcccggcaacaattaat |
| | | agactggatggaggcggataaagttgcaggaccacttctgcgctcggc |
| | | ccttccggctggctggtttattgctgataaatctggagccggtgagcg |
| | | tgggtctcgcggtatcattgcagcactggggccagatggtaagccctc |
| | | ccgtatcgtagttatctacacgacggggagtcaggcaactatggatga |
| | | acgaaatagacagatcgctgagataggtgcctcactgattaagcattg |
| | | gtaactgtcagaccaagtttactcatatatactttagattgatttaaa |
| | | acttcatttttaatttaaaaggatctaggtgaagatcctttttgataa |
| | | tctcatgaccaaaatcccttaacgtgagttttcgttccactgagcgtc |
| | | agaccccgtagaaaagatcaaaggatcttcttgagatcctttttttct |
| | | gcgcgtaatctgctgcttgcaaacaaaaaaaccaccgctaccagcggt |
| | | ggtttgtttgccggatcaagagctaccaactctttttccgaaggtaac |
| | | tggcttcagcagagcgcagataccaaatactgtccttctagtgtagcc |
| | | gtagttaggccaccacttcaagaactctgtagcaccgcctacatacct |
| | | cgctctgctaatcctgttaccagtggctgctgccagtggcgataagtc |
| | | gtgtcttaccgggttggactcaagacgatagttaccggataaggcgca |
| | | gcggtcgggctgaacggggggttcgtgcacacagcccagcttggagcg |
| | | aacgacctacaccgaactgagatacctacagcgtgagctatgagaaag |
| | | cgccacgcttcccgaagggagaaaggcggacaggtatccggtaagcgg |
| | | cagggtcggaacaggagagcgcacgagggagcttccagggggaaacgc |
| | | ctggtatctttatagtcctgtcgggtttcgccacctctgacttgagcg |
| | | tcgatttttgtgatgctcgtcaggggggcggagcctatggaaaaacgc |
| | | cagcaacgcggcctttttacggttcctggccttttgctggccttttgc |
| | | tcacatgttctttcctgcgttatcccctgattctgtggataaccgtat |
| | | taccgcctttgagtgagctgataccgctcgccgcagccgaacgaccga |
| | | gcgcagcgagtcagtgagcgaggaagcggaagagcgcccaatacgcaa |
| | | accgcctctccccgcgcgttggccgattcattaatgcagcag |
| 2 | Modified IL-1Ra insert | atggagatcagacgcagaagcgtgcgccacctgatcagcctgctgctg |
| | | ttcctgctgtacagcgagaccgcctgccaccccctgggcaagaggccc |
| | | tgcaagatgcaggccttcagaatctgggacgtgaatcagaaaaccttc |
| | | tacatgcgcaacaatcagctggtcgctggctacctgcaggagagcaac |
| | | accaagctgcaagagaagatcgacgtggtgcccatcgagcccgacgcc |
| | | ctgttcctgggcctgcacggcagaaagctgtgcctggcctgcgtgaag |
| | | tccggcgacgagatcagattccagctggaggccgtgaacatcaccgac |
| | | ctgagcaagaacaaggaggagaacaagcgcttcaccttcatcagaagc |
| | | aacagcggccctaccacctccttcgagagcgccgcctgccccggctgg |
| | | ttcctgtgcaccgcccaggaggccgacagacctgtctccctgaccaac |
| | | aagcccaaagaatccttcatggtgaccaagttctacctgcaagaggat |
| | | cagtga |
| 3 | Modified IL-1Ra insert | atggagatcagacgcagaagcgtgcgccacctgatcagcctgctgctg |
| | (98% identical to SEQ ID | ttcctgctgtatagcgagacagcctgccaccccctgggcaagaggccc |
| | NO: 2; bold letters are | tgcaagatgcaggccttcagaatctgggacgtgaatcagaaaaccttc |
| | nucleotide substitutions | tacatgcgcaacaatcagctggtcgctggctacctgcaggagagtaac |
| | within a codon (codon is | accaagctgcaagagaagatcgacgtggtgcccatcgagcccgacgcc |
| | underlined)) | ctgttcctgggcctgcacggcagaaagctgtgtctggcttgcgtgaag |
| | | tccggcgacgagatcagattccaactggaagccgtgaacatcaccgac |
| | | ctgagcaaaaacaaggaggagaacaagcgcttcaccttcatcagaagc |
| | | aacagtggccctaccacctccttcgagagcgccgcctgccccggctgg |
| | | ttcctgtgcaccgcccaggaggccgacagacctgtctccctgaccaac |
| | | aagcccaaagaatccttcatggtgaccaagttctacctgcaagaggat |
| | | caatga |
| 4 | Modified IL-1Ra insert | atggagatcagacgcagaagcgtgcgccacctgatcagcctgctgctg |
| | (99% identical to SEQ ID | ttcctgctgtatagcgagacagcctgccaccccctgggcaagaggccc |
| | NO: 2; bold letters are | tgcaagatgcaggccttcagaatctgggacgtgaatcagaaaaccttc |
| | nucleotide substitutions | tacatgcgcaacaatcagctggtcgctggctacctgcaggagagtaac |
| | within a codon (codon is | accaagctgcaagagaagatcgacgtggtgcccatcgagcccgacgcc |
| | underlined)) | ctgttcctgggcctgcacggcagaaagctgtgcctggcctgcgtgaag |
| | | tccggcgacgagatcagattccagctggaggccgtgaacatcaccgac |
| | | ctgagcaagaacaaggaggagaacaagcgcttcaccttcatcagaagc |
| | | aacagtggccctaccacctccttcgagagcgccgcctgccccggctgg |
| | | ttcctgtgcaccgcccaggaggccgacagacctgtctccctgaccaac |
| | | aagcccaaagaatccttcatggtgaccaagttctacctgcaagaggat |
| | | caatga |
| 5 | Modified IL-1Ra insert | atggagataagacgcagaagcgtacgccacctgatcagcctcctgctt |
| | (95% identical to SEQ ID | ttcctgctgtatagcgagacagcctgccatcccctgggcaagaggccc |
| | NO: 2; bold letters are | tgcaagatgcaggccttcagaatctgggacgtgaatcagaaaaccttc |
| | nucleotide substitutions | tacatgcgcaacaatcagcttgtcgctggatacctgcaggagagtaac |
| | within a codon (codon is | accaagctgcaagaaaagatcgacgtggtgcccatcgagcccgacgcc |
| | underlined)) | ctcttcctgggcctgcacggcagaaagctgtgtctggcttgcgtgaag |
| | | tccggcgacgagatcagattccaactggaagccgtgaacatcaccgac |
| | | ctgagcaaaaacaaggaggagaacaagcgcttcaccttcatcaggagc |
| | | aacagtggccctaccacctcattcgagagcgccgcctgtcccggctgg |
| | | ttcctgtgcaccgcccaggaggccgacagacccgtctccctgaccaat |
| | | aagcccaaagaatcattcatggtgaccaagttctacctacaagaggat |
| | | caatga |

In some embodiments, the IL-1Ra peptide encoded by the IL-1Ra insert comprises IL-1Ra (see SEQ ID NO: 6, SEQ ID NO: 7 in Table 2 below).

TABLE 2

| SEQ ID | | |
|---|---|---|
| NO: | DESCRIPTION | SEQUENCE |
| 6 | IL-1Ra | MEIRRRSVRH LISLLLFLFY |
| | (UNIPROT | SETACHPLGK PCKMQAFRI |
| | O18999) | WDVNQKTFYM RNNQLVAGYL |
| | | QESNTKLQEK IDVVPIEPDA |
| | | LFLGLHGRKL CLACVKSGDE |
| | | IRFQLEAVNI TDLSKNKEEN |
| | | KRFTFIRSNS GPTTSFESAA |
| | | CPGWFLCTAQ EADRPVSLTN |
| | | KPKESFMVTK FYLQEDQ |
| 7 | IL-1Ra | MEIRRRSVRH LISLLLFLLY |
| | (encoded | SETACHPLGK PCKMQAFRI |
| | by SEQ | WDVNQKTFYM RNNQLVAGYL |
| | ID NO: 2) | QESNTKLQEK IDVVPIEPDA |
| | | LFLGLHGRKL CLACVKSGDE |
| | | IRFQLEAVNI TDLSKNKEEN |
| | | KRFTFIRSNS GPTTSFESAA |
| | | CPGWFLCTAQ EADRPVSLTN |
| | | KPKESFMVTK FYLQEDQ |

The transgene (e.g., nucleotide sequence encoding protein of interest) is operably linked to a promoter. In some embodiments, the promoter comprises the cytomegalovirus (CMV) promoter. The present invention is not limited to the CMV promoter and may feature any appropriate promoter or portions of various promoters. Examples of promoters include CMV promoter, hybrid CMV promoter, CAG promoter, human beta-actin promoter, hybrid beta-actin promoter, EF1 promoter, U1a promoter, U1b promoter, a Tet-inducible promoter, a VP16-LexA promoter, chicken beta-actin (CBA) promoter, human elongation factor-1alpha promoter, simian virus 40 (SV40) promoter, and herpes simplex virus thymidine kinase promoter. In some embodiments, the promoter comprises a hybrid promoter. As an example, Table 3 shows an IL-1 beta/IL-6 hybrid promoter (see also van de Loo et al., 2004, Gene Therapy 11:581-590). The present invention is also not limited to the hybrid promoter shown in Table 3.

TABLE 3

| SEQ | | |
|---|---|---|
| ID | | |
| NO: | DESCRIPTION | SEQUENCE |
| 8 | IL-1 beta/ | atccaagag ggagaagaag cccattggag |
| | IL-6 hybrid | atgatgccat aaaggaagtg gaagcgatat |
| | promoter | gataaaaatc atagtgccca ttcccaaata |
| | | atcccagaag cagaagggaa aggagagaaa |
| | | tatccacaaa gacaggtgtg ggtacacaca |
| | | acatttttca tactttaaga tcccagagga |
| | | ctcatggaaa tgatacaaga aaatgactca |
| | | taagaacaaa tattaggaag ccagtgccaa |
| | | gaatgagatg ggaaattggg gaaaatgttg |
| | | ggggcagatt gcttagttct gttctaagca |
| | | agagggtgaa caaggaagga acagctcact |
| | | acaaagaaca gacatcactg catgtacaca |
| | | caataatata agaactaacc catgattatt |
| | | ttgcttgtct tcttgttcaa aatgattgaa |
| | | gaccaatgag atgagatcaa ccttgataac |
| | | tggctggctt cggcatgatt agacacaaga |
| | | tggtatcagg gcacttgctg ctttgaataa |
| | | tgtcagtctc ctgtcttgga agaatgacct |
| | | gacagggtaa agaggaactt gcagctgaga |
| | | aaggctttag tgactcaaga gctgaataat |
| | | tccccaaaag ctggagcatc ctggcatttc |
| | | cagctcccca tctctgcttg ttccacttcc |
| | | ttggggctac atcaccatct acatcatcat |
| | | cactcttcca ctccctccct tagtgccaac |
| | | tatgtttata gcgagatatt ttctgctcat |
| | | tggggatcgg aaggaagtgc tgtggcctga |
| | | gcggtctcct tgggaagaca ggatctgata |
| | | catacgttgc acaacctatt tgacataaga |
| | | ggtttcactt cctgagatgg atgggatggt |
| | | agcagatttg ggtccaggtt acagggccag |
| | | gatgagacat ggcagaactg tggagactgt |
| | | tacgtcaggg ggcattgccc catggctcca |
| | | aaatttccct cgagc ctctggccc |
| | | caccctcacc ctccaacaaa gatttatcaa |
| | | atgtgggatt ttcccatgag tctcaatatt |
| | | agagtctcaa cccccaataa atataggact |
| | | ggagatgtct gaggctcatt ctgccctcga |
| | | gcccaccggg aacgaaagag aagctctatc |
| | | tcccctccag gagcccagct atgaactcct |
| | | tc |

In some embodiments, the sc-rAAV vector is packaged within a capsid. In some embodiments, the capsid comprises at least a portion of AAV serotype 1 (AAV1), AAV serotype 2, (AAV2), AAV serotype 3, (AAV3), AAV serotype 4, (AAV4), AAV serotype 5, (AAV5), AAV serotype 6, (AAV6), derivatives thereof, or combination thereof. For example, in some embodiments, the capsid comprises at least a portion of AAV serotype 2 and at least a portion of AAV serotype 6, e.g., AAV2.5.

The composition, e.g., the composition comprising the sc-rAAV, may be introduced into cells in a location of interest (e.g., in a horse). For example, in some embodiments when treating symptoms of osteoarthritis, the composition may be introduced into cells (e.g., chondrocytes, synoviocytes, e.g., type A, type B, etc.) in a joint via direct intraarticular injection. In some embodiments, the composition is administered to a joint, synovium, subsynovium, joint capsule, tendon, ligament, cartilage, or peri-articular muscle of the horse. The present invention is not limited to the aforementioned conditions (e.g., osteoarthritis), the means of administration (e.g., intraarticular injection), the location of interest (e.g., joint), or cell type (e.g., chondrocytes, synoviocytes). For example, in some embodiments, other cell types that may be transduced may include mesenchymal stem cells.

The sc-rAAV transduces the vector into cells and the modified IL-1Ra peptide is expressed. In some embodiments, the IL-1Ra peptide is expressed so as to provide the horse with a therapeutically effective amount of said modified IL-1Ra peptide effective for ameliorating symptoms associated with various conditions such as osteoarthritis.

In some embodiments, introduction of the composition (e.g., the sc-rAAV) is performed once. In some embodiments, introduction of the composition (e.g., the sc-rAAV) is performed twice, e.g., a first time and a second time subsequent to the first time. In some embodiments, introduction of the composition is performed more than two times, e.g., three times, four times, five times, etc. The introduction of the composition a second time may be performed at a time point after the time when the method is first performed, e.g., after 3 months, 4 months, 5 months, 6 months, 7 months, 8 months, 9 months, 10 months, 11 months, 1 year, more than one year, etc.

The composition may comprise any appropriate pharmaceutical composition. In some embodiments, the composition comprises a buffered solution. In some embodiments, the buffered solution comprises phosphate buffered saline (PBS). In some embodiments, the composition further comprises sorbitol, e.g., 5% sorbitol. In some embodiments, the composition further comprises a salt, e.g., NaCl. The concentration of salt may be any appropriate concentration, e.g., 350 mM NaCl, more than 350 mM NaCl, less then 350 mM, etc.

In some embodiments, the composition (e.g., the sc-rAAV) is co-administered with a secondary therapy. In some embodiments, the secondary therapy comprises a therapeutic for OA or RA or any other appropriate therapy for treating the symptoms of the condition. Non-limiting examples of secondary therapies for OA include glucocorticoids, hyaluronan (viscosupplementation), platelet-rich plasma, and recombinant, human IL-1Ra (Anakinra; Kineret®). For example, in some embodiments, the sc-rAAV is co-administered with glucocorticoids or platelet-rich plasma.

The disclosures of the following U.S. Patents are incorporated in their entirety by reference herein: US2008/0187576, US2009/0104155, KR2012041139, JP2015518816, WO2013151672, WO2008088895, U.S. Pat. Nos. 8,529,885, 7,037,492, US20070128177, U.S. Pat. Nos. 6,491,907, 8,999,948, US20150218586, U.S. Pat. No. 7,892,824, US20130295614, JP2002538770, JP2010516252, KR2002027450, KR2003028080, U.S. Pat. No. 6,482,634, US20090105148, US20120232130, US20140234255, U.S. Pat. Nos. 5,756,283, 6,083,716, WO2002038782, WO2007039699, WO2012047093, WO2014170470, WO2015018860, WO2015044292, WO2015158749, U.S. Pat. Nos. 7,452,696, 6,943,153, 6,429,001, WO2015031392, WO2004092211.

### Example 1

Example 1 describes administration of a sc-rAAV of the present invention (encoding IL-1Ra). The present invention is not limited to the disclosure of Example 1.

Fifteen horses diagnosed with osteoarthritis in the knee are used for a clinical trial investigating administration of a sc-rAAV (encoding IL-1Ra) of the present invention. Twelve of the fifteen horses are administered the sc-rAAV via intraarticular injection into the knee with osteoarthritis at 1×10¹⁰ viral genes per knee. The remaining three horses are treated with a vehicle control. After three weeks, all horses are evaluated for lameness. At the three-month time point, the three control animals are given a second treatment of vehicle control. Three of the 12 remaining previously treated horses are administered a second administration of the same sc-rAAV (via intraarticular injection into the knee with osteoarthritis at 1×10¹⁰ viral genes per knee); three are administered a second administration of a sc-rAAV (encoding IL-1RA) of the present invention that is different from the first sc-rAAV (via intraarticular injection into the knee with osteoarthritis at 1×10¹⁰ viral genes per knee); three are administered a second administration of the same sc-rAAV (via intraarticular injection into the knee with osteoarthritis at 1×10¹⁰ viral genes per knee) in combination with a secondary therapy (e.g., glucocorticoids and platelet-rich plasma); and the remaining three are administered a second administration of the sc-rAAV (via intraarticular injection) into the knee with osteoarthritis at 1×10¹⁰ viral genes per knee) in combination with an immunosuppressant. The horses are evaluated for lameness at 3, 6, and 9 weeks post-administration (of the second administrations).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described embodiments of the present invention, it will be readily apparent to those skilled in the art that modifications may be made thereto which do not exceed the scope of the appended claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

Any reference numbers recited in the below claims are solely for ease of examination of this patent application, and are exemplary, and are not intended in any way to limit the scope of the claims to the particular features having the corresponding reference numbers in the drawings.

What is claimed is:

1. A method of delivering a therapeutically effective amount of interleukin-1 receptor agonist (IL-1Ra) peptide to a horse in need thereof, said method comprising: introducing into a location of interest a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene is at least 95% identical SEQ ID NO: 2;
wherein the sc-rAAV transduces the vector into cells in the location of interest, wherein the modified IL-1Ra gene is expressed so as to provide the horse with the therapeutically effective amount of said IL-1Ra peptide.

2. The method of claim 1, wherein said horse is diagnosed with or is at risk for developing osteoarthritis.

3. The method of claim 1, wherein the location of interest is a joint, synovium, subsynovium, joint capsule, tendon, ligament, cartilage, or peri-articular muscle of the horse.

4. The method of claim 1, wherein the composition is introduced into the location of interest via direct intraarticular injection

5. The method of claim 1, wherein the cells are chondrocytes, synoviocytes, or a combination thereof.

6. The method of claim 1, wherein the method is performed a second time at a time point after a time when the method is performed first.

7. The method of claim 1, wherein the time point is at least 3 months.

8. The method of claim 1, wherein the method further comprises co-introducing a secondary therapy to the location of interest in combination with the composition.

9. The method of claim 8, wherein the secondary therapy comprises a glucocorticoid, hyaluronan, platelet-rich plasma, recombinant, horse IL-1Ra, or a combination thereof.

10. The method of claim 1, wherein the promoter comprises a CMV promoter.

11. The method of claim 1, wherein the engineered capsid comprises at least a portion of serotype AAV2 and at least a portion of serotype AAV6.

12. The method of claim 1, wherein the engineered capsid comprises at least a portion of serotype AAV1, AAV2, AAV3, AAV4, AAV5, AAV6, AAV7, AAV8, AAV9, AAV10, AAV11, or a combination thereof.

13. The method of claim 1, wherein the vector further comprises SV40 and bovine growth hormone (bGH) polyadenylation sequences.

14. The method of claim 13, wherein the vector further comprises SV40 splice donor (SD) and splice acceptor (SA) sites.

15. The method of claim 1, wherein the vector comprises sc-rAAV2.5Hu-IL-1Ra.

16. A method of ameliorating symptoms of osteoarthritis in a horse, said method comprising introducing into a location of interest a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene is at least 95% identical to SEQ ID NO: 2;
wherein the sc-rAAV transduces the vector into cells in the location of interest, wherein the modified IL-1Ra gene is expressed so as to provide the horse with an amount of IL-1Ra peptide effective for ameliorating symptoms associated with osteoarthritis.

17. The method of claim 16, wherein the location of interest is a joint, synovium, subsynovium, joint capsule, tendon, ligament, cartilage, or peri-articular muscle of the horse.

18. The method of claim 16, wherein the composition is introduced into the location of interest via direct intraarticular injection

19. The method of claim 16, wherein the cells are chondrocytes, synoviocytes, or a combination thereof.

20. The method of claim 16, wherein the method is performed a second time at a time point after a time when the method is performed first.

21. The method of claim 16, wherein the time point is at least 3 months.

22. The method of claim 16, wherein the method further comprises co-introducing a secondary therapy to the location of interest in combination with the composition.

23. The method of claim 22, wherein the secondary therapy comprises a glucocorticoid, hyaluronan, platelet-rich plasma, recombinant, horse IL-1Ra, or a combination thereof.

24. The method of claim 16, wherein the promoter comprises a CMV promoter.

25. The method of claim 16, wherein the engineered capsid comprises at least a portion of serotype AAV2 and at least a portion of serotype AAV6.

26. The method of claim 16, wherein the engineered capsid comprises at least a portion of serotype AAV1, AAV2, AAV3, AAV4, AAV5, AAV6, AAV7, AAV8, AAV9, AAV10, AAV11, or a combination thereof.

27. The method of claim 16, wherein the vector further comprises SV40 and bovine growth hormone (bGH) polyadenylation sequences.

28. The method of claim 27, wherein the vector further comprises SV40 splice donor (SD) and splice acceptor (SA) sites.

29. The method of claim 16, wherein the vector comprises sc-rAAV2.5Hu-IL-1Ra.

30. A method of delivering IL-1Ra peptide to a chondrocyte or synoviocyte, said method comprising contacting the chondrocyte or synoviocyte with a recombinant self-complementary adeno-associated virus (sc-rAAV) comprising:
a. an engineered adeno-associated virus (AAV) capsid comprising at least a portion of serotype 2 and at least a portion of serotype 6; and
b. a vector packaged within the capsid, said vector comprising a modified IL-1Ra gene operably linked to a CMV promoter, the modified IL-1Ra gene is at least 95% identical to SEQ ID NO: 2;
wherein the sc-rAAV transduces the vector into the chondrocyte or synoviocyte and the modified IL-1Ra gene is expressed to as to provide IL-1Ra peptide to the chondrocyte or synoviocyte.

31. The method of claim 30, wherein the vector comprises sc-rAAV2.5Hu-IL-1Ra.

32. The method of claim 30, wherein the vector further comprises SV40 and bovine growth hormone (bGH) polyadenylation sequences.

33. The method of claim 30, wherein the vector further comprises SV40 splice donor (SD) and splice acceptor (SA) sites.

34. A composition comprising a recombinant self-complementary adeno-associated virus (sc-rAAV), wherein said sc-rAAV comprises:
a. an engineered capsid comprising at least a portion of serotype 2 and at least a portion of serotype 6; and
b. a vector packaged within the capsid, said vector comprises a nucleic acid sequence encoding a modified IL-1Ra peptide operably linked to a CMV promoter, the nucleic acid sequence that encodes the modified IL-1Ra peptide is at least 90% identical to SEQ ID NO: 2;

35. The composition of claim 34, wherein the vector further comprises SV40 and bovine growth hormone (bGH) polyadenylation sequences.

36. The composition of claim 35, wherein the vector further comprises SV40 splice donor (SD) and splice acceptor (SA) sites.

37. The composition of claim 34, wherein the vector comprises sc-rAAV2.5Hu-IL-1Ra.

38. A recombinant self-complementary adeno-associated virus (sc-rAAV) vector comprising a modified IL-1Ra gene operably linked to a CMV promoter, the modified IL-1Ra gene is at least 95% identical to SEQ ID NO: 2.

39. The vector of claim 38 further comprising SV40 and bovine growth hormone (bGH) polyadenylation sequences.

40. The vector of claim 39 further comprising SV40 splice donor (SD) and splice acceptor (SA) sites.

41. The vector of claim 38 comprising sc-rAAV2.5Hu-IL-1Ra.

42. A method of repairing cartilage in a horse in need thereof, said method comprising: introducing into a location of cartilage a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene is at least 95% identical SEQ ID NO: 2;
wherein the sc-rAAV transduces the vector into cells in the location of cartilage, wherein the modified IL-1Ra gene is expressed so as to provide the horse with IL-1Ra peptide effective for repairing cartilage.

43. The method of claim 42, wherein said horse is diagnosed with or is at risk for developing osteoarthritis.

44. The method of claim 42, wherein the composition is introduced into the location of cartilage via direct intraarticular injection

45. The method of claim 42, wherein the cells are chondrocytes, synoviocytes, or a combination thereof.

46. The method of claim 42, wherein the method is performed a second time at a time point after a time when the method is performed first.

47. The method of claim 42, wherein the time point is at least 3 months.

48. The method of claim 42, wherein the method further comprises co-introducing a secondary therapy to the location of cartilage in combination with the composition.

49. The method of claim 48, wherein the secondary therapy comprises a glucocorticoid, hyaluronan, platelet-rich plasma, recombinant, horse IL-1Ra, or a combination thereof.

50. The method of claim 42, wherein the promoter comprises a CMV promoter.

51. The method of claim 42, wherein the engineered capsid comprises at least a portion of serotype AAV2 and at least a portion of serotype AAV6.

52. The method of claim 42, wherein the engineered capsid comprises at least a portion of serotype AAV1, AAV2, AAV3, AAV4, AAV5, AAV6, AAV7, AAV8, AAV9, AAV10, AAV11, or a combination thereof.

53. The method of claim 42, wherein the vector comprises sc-rAAV2.5Hu-IL-1Ra.

54. A method of providing interleukin-1 receptor agonist (IL-1Ra) peptide to an area of inflammation, said method comprising: introducing into a location of inflammation a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene is at least 95% identical SEQ ID NO: 2;
wherein the sc-rAAV transduces the vector into cells in the location of inflammation, wherein the modified IL-1Ra gene is expressed so as to provide the cells in the location of inflammation a therapeutically effective amount of IL-1Ra peptide effective for reducing inflammation.

55. The method of claim 54, wherein the location of inflammation is a joint, synovium, subsynovium, joint capsule, tendon, ligament, cartilage, or peri-articular muscle of the horse.

56. The method of claim 54, wherein the composition is introduced into the location of inflammation via direct intraarticular injection

57. The method of claim 54, wherein the cells are chondrocytes, synoviocytes, or a combination thereof.

58. The method of claim 54, wherein the promoter comprises a CMV promoter.

59. The method of claim 54, wherein the engineered capsid comprises at least a portion of serotype AAV2 and at least a portion of serotype AAV6.

60. The method of claim 54, wherein the engineered capsid comprises at least a portion of serotype AAV1, AAV2, AAV3, AAV4, AAV5, AAV6, AAV7, AAV8, AAV9, AAV10, AAV11, or a combination thereof.

61. The method of claim 54, wherein the vector comprises sc-rAAV2.5Hu-IL-1Ra.

62. A method of providing a horse in need thereof a therapeutically effective amount of interleukin-1 receptor agonist (IL-1Ra), said method comprising: introducing into a location of interest a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene encodes IL-1Ra according to SEQ ID NO: 6 or SEQ ID NO: 7;
wherein the sc-rAAV transduces the vector into cells in the location of interest, wherein IL-1Ra is expressed so as to provide the horse with the therapeutically effective amount of said IL-1Ra.

63. A method of ameliorating symptoms of osteoarthritis in a horse, said method comprising introducing into a location of interest a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene encodes IL-1Ra according to SEQ ID NO: 6 or SEQ ID NO: 7;
wherein the sc-rAAV transduces the vector into cells in the location of interest, wherein IL-1Ra expressed so as to provide the horse with an amount of IL-1Ra effective for ameliorating symptoms associated with osteoarthritis.

64. A method of delivering IL-1Ra peptide to a chondrocyte or synoviocyte, said method comprising contacting the chondrocyte or synoviocyte with a recombinant self-complementary adeno-associated virus (sc-rAAV) comprising:
a. an engineered adeno-associated virus (AAV) capsid comprising at least a portion of serotype 2 and at least a portion of serotype 6; and
b. a vector packaged within the capsid, said vector comprising a modified IL-1Ra gene operably linked to a CMV promoter, the modified IL-1Ra encodes IL-1Ra according to SEQ ID NO: 6 or SEQ ID NO: 7;
wherein the sc-rAAV transduces the vector into the chondrocyte or synoviocyte and IL-1Ra is expressed to as to provide IL-1Ra to the chondrocyte or synoviocyte.

65. A composition comprising a recombinant self-complementary adeno-associated virus (sc-rAAV), wherein said sc-rAAV comprises:
a. an engineered capsid comprising at least a portion of serotype 2 and at least a portion of serotype 6; and
b. a vector packaged within the capsid, said vector comprises a nucleic acid sequence encoding a modified IL-1Ra peptide operably linked to a CMV promoter, the nucleic acid sequence encodes IL-1Ra according to SEQ ID NO: 6 or SEQ ID NO: 7.

66. A method of repairing cartilage in a horse in need thereof, said method comprising: introducing into a location of cartilage a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene encodes IL-1Ra according to SEQ ID NO: 6 or SEQ ID NO: 7;
wherein the sc-rAAV transduces the vector into cells in the location of cartilage, wherein IL-1Ra is expressed so as to provide the horse with IL-1Ra effective for repairing cartilage.

67. A method of providing interleukin-1 receptor agonist (IL-1Ra) peptide to an area of inflammation, said method comprising: introducing into a location of inflammation a composition comprising a recombinant self-complementary adeno-associated virus (sc-rAAV), wherein said sc-rAAV comprises:
a. an engineered AAV capsid; and
b. a vector packaged within the capsid, said vector comprising a modified IL-1Ra gene operably linked to a promoter, the modified IL-1Ra gene encodes IL-1Ra according to SEQ ID NO: 6 or SEQ ID NO: 7;
wherein the sc-rAAV transduces the vector into cells in the location of inflammation, wherein IL-1Ra is expressed so as to provide the cells in the location of inflammation a therapeutically effective amount of IL-1Ra effective for reducing inflammation.


SEQUENCE LISTING (8 sequences)

SEQ ID NO: 1 - DNA, 5754 bp, Artificial Sequence
  Equine Optimized IL-1ra p-trs-KS Expression Vector Sequence with CMV promotor & ampicillin resistance
ctgcgcgctcgctcgctcactgaggccgcccgggcaaagcccgggcgtcgggcgacctttggtcgcccggcctcagtgagcgagcgagcgcgcagagagggagtggggttcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccggtaccggtgccaccatggagatcagacgcagaagcgtgcgccacctgatcagcctgctgctgttcctgctgtacagcgagaccgcctgccaccccctgggcaagaggccctgcaagatgcaggccttcagaatctgggacgtgaatcagaaaaccttctacatgcgcaacaatcagctggtcgctggctacctgcaggagagcaacaccaagctgcaagagaagatcgacgtggtgcccatcgagcccgacgccctgttcctgggcctgcacggcagaaagctgtgcctggcctgcgtgaagtccggcgacgagatcagattccagctggaggccgtgaacatcaccgacctgagcaagaacaaggaggagaacaagcgcttcaccttcatcagaagcaacagcggccctaccacctccttcgagagcgccgcctgccccggctggttcctgtgcaccgcccaggaggccgacagacctgtctccctgaccaacaagcccaaagaatccttcatggtgaccaagttctacctgcaagaggatcagtgagcggccgcgggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggctggggagagatcctagtttccatgctctaggagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 2 - DNA, 534 bp, Artificial Sequence
  Modified IL-1Ra insert
atggagatcagacgcagaagcgtgcgccacctgatcagcctgctgctgttcctgctgtacagcgagaccgcctgccaccccctgggcaagaggccctgcaagatgcaggccttcagaatctgggacgtgaatcagaaaaccttctacatgcgcaacaatcagctggtcgctggctacctgcaggagagcaacaccaagctgcaagagaagatcgacgtggtgcccatcgagcccgacgccctgttcctgggcctgcacggcagaaagctgtgcctggcctgcgtgaagtccggcgacgagatcagattccagctggaggccgtgaacatcaccgacctgagcaagaacaaggaggagaacaagcgcttcaccttcatcagaagcaacagcggccctaccacctccttcgagagcgccgcctgccccggctggttcctgtgcaccgcccaggaggccgacagacctgtctccctgaccaacaagcccaaagaatccttcatggtgaccaagttctacctgcaagaggatcagtga

SEQ ID NO: 3 - DNA, 534 bp, Artificial Sequence
  Modified IL-1Ra insert  (98% identical to SEQ ID NO 2)
atggagatcagacgcagaagcgtgcgccacctgatcagcctgctgctgttcctgctgtatagcgagacagcctgccaccccctgggcaagaggccctgcaagatgcaggccttcagaatctgggacgtgaatcagaaaaccttctacatgcgcaacaatcagctggtcgctggctacctgcaggagagtaacaccaagctgcaagagaagatcgacgtggtgcccatcgagcccgacgccctgttcctgggcctgcacggcagaaagctgtgtctggcttgcgtgaagtccggcgacgagatcagattccaactggaagccgtgaacatcaccgacctgagcaaaaacaaggaggagaacaagcgcttcaccttcatcagaagcaacagtggccctaccacctccttcgagagcgccgcctgccccggctggttcctgtgcaccgcccaggaggccgacagacctgtctccctgaccaacaagcccaaagaatccttcatggtgaccaagttctacctgcaagaggatcaatga

SEQ ID NO: 4 - DNA, 534 bp, Artificial Sequence
  Modified IL-1Ra insert  (99% identical to SEQ ID NO 2)
atggagatcagacgcagaagcgtgcgccacctgatcagcctgctgctgttcctgctgtatagcgagacagcctgccaccccctgggcaagaggccctgcaagatgcaggccttcagaatctgggacgtgaatcagaaaaccttctacatgcgcaacaatcagctggtcgctggctacctgcaggagagtaacaccaagctgcaagagaagatcgacgtggtgcccatcgagcccgacgccctgttcctgggcctgcacggcagaaagctgtgcctggcctgcgtgaagtccggcgacgagatcagattccagctggaggccgtgaacatcaccgacctgagcaagaacaaggaggagaacaagcgcttcaccttcatcagaagcaacagtggccctaccacctccttcgagagcgccgcctgccccggctggttcctgtgcaccgcccaggaggccgacagacctgtctccctgaccaacaagcccaaagaatccttcatggtgaccaagttctacctgcaagaggatcaatga

SEQ ID NO: 5 - DNA, 534 bp, Artificial Sequence
  Modified IL-1Ra insert  (95% identical to SEQ ID NO 2
atggagataagacgcagaagcgtacgccacctgatcagcctcctgcttttcctgctgtatagcgagacagcctgccatcccctgggcaagaggccctgcaagatgcaggccttcagaatctgggacgtgaatcagaaaaccttctacatgcgcaacaatcagcttgtcgctggatacctgcaggagagtaacaccaagctgcaagaaaagatcgacgtggtgcccatcgagcccgacgccctcttcctgggcctgcacggcagaaagctgtgtctggcttgcgtgaagtccggcgacgagatcagattccaactggaagccgtgaacatcaccgacctgagcaaaaacaaggaggagaacaagcgcttcaccttcatcaggagcaacagtggccctaccacctcattcgagagcgccgcctgtcccggctggttcctgtgcaccgcccaggaggccgacagacccgtctccctgaccaataagcccaaagaatcattcatggtgaccaagttctacctacaagaggatcaatga

SEQ ID NO: 6 - protein, 176 aa, Equus caballus
MEIRRRSVRHLISLLLFLFYSETACHPLGKPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 7 - protein, 176 aa, Artificial Sequence
  IL-1Ra encoded by SEQ ID NO2
MEIRRRSVRHLISLLLFLLYSETACHPLGKPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 8 - DNA, 1165 bp, Artificial Sequence
  IL-1 beta/ IL-6 hybrid promoter
atccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagaggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctggcttcggcatgattagacacaagatggtatcagggcacttgctgctttgaataatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttc